(19) United States
(12) Patent Application Publication
(10) Pub. No.: US 2019/0133965 A1
(43) Pub. Date: May 9, 2019

(54) PHYTOTERPENOID FACILITATION OF THERAPEUTIC ONSET AND EFFICACY

(71) Applicant: JC Pharma Inc., Boulder, CO (US)

(72) Inventors: Jason Allen Cranford, Boulder, CO (US); Donald Channing Cooper, Boulder, CO (US)

(73) Assignee: JC Pharma Inc., Boulder, CO (US)

(21) Appl. No.: 16/240,475

(22) Filed: Jan. 4, 2019

Related U.S. Application Data
(63) Continuation-in-part of application No. 15/867,240, filed on Jan. 10, 2018.
(60) Provisional application No. 62/445,047, filed on Jan. 11, 2017.

(51) Int. Cl. A61K 31/015; A61K 31/05; A61K 31/352; A61K 36/185; A61K 9/00; A61K 47/44; A61P 25/00

(52) U.S. Cl. CPC A61K 31/015; A61K 31/05; A61K 31/352; A61P 25/00; A61K 9/006; A61K 47/44; A61K 36/185

(57) ABSTRACT

Embodiments described are generally directed to a method for applying a composition for managing dysallostasis effects to cannabinoids from repeated administration. The method envisions providing an anti-dysallostatic composition essentially comprised of a combination of phytoterpenoid and phytocannabinoid plus an independently derived phytoterpenoid that is between 70% and 95%, the phytocannabinoid includes THC and CBD. A dose of the composition is introduced to a subject sublingually while in a state of dysallostasis from prior consumption of the THC, the dose is approximately 5 mg of the THC, 1.25 mg of the CBD and 0.3-0.6 mg of the phytoterpenoid and between approximately 0.3-2.0 mg of the independently derived phytoterpenoid. The dose is increased if there is a change from the state of dysallostasis to two doses at a time interval, but if there is not a change from the state of dysallostasis, increasing administration of the composition sublingually to 3 doses at the predetermined interval.

### CROSS-REFERENCE TO RELATED APPLICATIONS

This application is a Continuation-In-Part Application which claims priority to and the benefit of U.S. patent application Ser. No. 15/867,240 entitled: PHYTOTERPENOID FACILITATION OF THERAPEUTIC ONSET AND EFFICACY OF SUBLINGUAL CANNABINOID ADMINISTRATION, filed on Jan. 10, 2018, the entire disclosure of which is hereby incorporated by reference; U.S. patent application Ser. No. 15/867,240 which is a Non-Provisional application claiming priority to and the benefit of U.S. Provisional Patent Application No. 62/445,047 entitled: CANNABIS BASED NEUROPROTECTION FOR GLUTAMATE EXCITOTOXICITY AND OXIDATIVE STRESS, filed on Jan. 11, 2017, the entire disclosure of which is hereby also incorporated by reference.

### FIELD OF THE INVENTION

The present invention relates generally to pharmaceutical compounds that mitigate dysallostasis effects of physiological responses to phytocannabinoids from repeated administration.

### BACKGROUND

Cannabis-based formulations have been used for centuries to treat a variety of medical conditions. Although there are dozens of molecular variants of cannabinoids in a typical cannabis plant, (−)-trans-Δ⁹-tetrahydrocannabinol (THC) and cannabidiol (CBD) are most commonly identified as providing medicinal benefits. It is known that the proven medical benefits of THC and CBD lose their therapeutic effect over a period of repeated uses. THC and CBD initially work well to manage symptoms of specific ailments referred to as a “honeymoon” (dysallostatic) effect, but over repeated uses lose their effectiveness.

It is to improvements in addressing the honeymoon effect that embodiments of the present invention are directed.

### SUMMARY

One embodiment described herein contemplates a chemical composition of phytoterpenoid ingredients that when combined with administration of exogenous THC and/or CBD cannabinoid pharmacological compounds with independently derived terpenoids to manage dysallostasis effects from repeated exposure to the exogenous THC and/or CBD.

One embodiment described herein contemplates a formulation to prevent dysallostasis effects of physiological responses to cannabinoids from repeated administration, the formulation comprising: a base vehicle and an anti-dysallostatic composition, the anti-dysallostatic composition comprising a first part phytoterpenoid and phytocannabinoid and a second part independently derived phytoterpenoid, the first part is between 70% and 95%.

Other embodiments envision a method for managing dysallostasis effects to cannabinoids from repeated administration, the method comprising: providing an anti-dysallostatic composition essentially comprised of a combination of phytoterpenoid and phytocannabinoid plus an independently derived phytoterpenoid, the combination of phytoterpenoid and phytocannabinoid is between 70% and 95%, the phytocannabinoid includes THC and CBD; introducing a dose of the composition sublingually, orally or nasally to a recipient in a state of dysallostasis from prior consumption of the THC, the dose is approximately 5 mg of the THC, 1.25 mg of the CBD and 0.3-0.6 mg of the phytoterpenoid and between approximately 0.3-2.0 mg of the independently derived phytoterpenoid; and if there is a change from the state of dysallostasis after the introducing step, increasing administration of the composition sublingually to two of the doses at a predetermined time interval, if there is not a change from the state of dysallostasis after the introducing step, increasing administration of the composition sublingually to 3 of the doses at the predetermined interval.

Yet other embodiments herein contemplate a formulation to prevent dysallostasis effects of physiological responses to cannabinoids from repeated administration, the formulation comprising: a dose of an anti-dysallostatic composition adapted to be administered to a recipient sublingually while the recipient is in a state of dysallostasis from prior consumption of THC, the dose is approximately 5 mg of the THC, 1.25 mg of the CBD, 0.3-0.6 mg of the phytoterpenoid and between approximately 0.3-2.0 mg of the independently derived terpenoid, the phytoterpenoid is essentially composed of beta-caryophyllene and myrcene; and a plant derived oil adapted to be a base vehicle for the formulation.

### BRIEF DESCRIPTION OF THE DRAWINGS

FIG. 1 illustratively depicts a pie chart of a base vehicle and an anti-dysallostatic composition consistent with embodiments of the present invention;

FIG. 2 illustratively depicts a pie chart of an anti-dysallostatic composition which includes a first part comprising phytoterpenoid and phytocannabinoid and a second part comprising an independently derived terpenoid consistent with embodiments of the present invention;

FIGS. 3A-3D illustratively depict chemical schematics of myrcene, beta-caryophyllene, CBD and THC as used with embodiments of the present invention; and

FIG. 4 illustratively depicts a block diagram of method steps using embodiments of an anti-dysallostatic composition consistent with embodiments of the present invention.

### DETAILED DESCRIPTION

Initially, the present disclosure is by way of example only, not by limitation. Thus, although the instrumentalities described herein are for the convenience of explanation, shown and described with respect to exemplary embodiments, it will be appreciated that the principles herein may be applied equally in other types of situations involving adding an independent source of phytoterpenoids to a composition of plant distilled phytoterpenoids and phytocannabinoids consistent with spirit and scope of the present invention.

Dysallostasis is the process by which the state of internal, physiological equilibrium cannot be maintained by an organism in response to sustained repeated physiological or pharmacological activation. As used herein, dysallostasis of a pharmacological effect on a human (or other living creature for that matter) is the persistent disruption in potency or efficacy over continued repeated exposure. This disruption at a fixed dose may be either a progressive loss of response (tolerance) or a progressive increase in response (sensitization) or a combination of both on different physiological processes. For example, a pharmacological agent (drug) may be efficacious or otherwise work well to treat a reported symptom when initially used, but after a series of administrations at the same dose loses its ability to treat the symptom in a satisfactory manner. Likewise, a drug dose may fail elicit a therapeutic response initially, but with repeated exposures may begin to elicit therapeutic responses to treat some symptoms, but not others. The emergence of “off target” or “side effects” is an example of dysallostasis. The current problem with the state of administration of cannabinoid compounds for medicinal treatments is the persistent dysallostatic adaptations to the physiological and psychological responses to the cannabinoid compounds (e.g., exogenous THC and/or CBD) when used by a human with an ailment. Once concrete example of dysallostasis is the newly described Cannabinoid Hyperemesis Syndrome (CHS). CHS is a condition that leads to repeated and severe bouts of vomiting that only occurs in daily long-term users of cannabis.

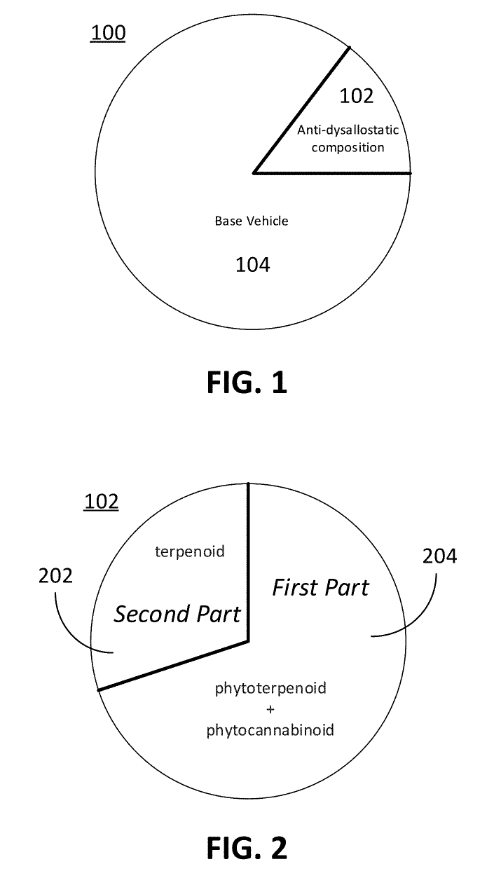

Certain embodiments contemplate a formulation to prevent dysallostasis effects of physiological responses to cannabinoids from repeat administrations consistent with embodiments of the present invention. As shown in FIG. 1, certain embodiments envision an anti-dysallostatic formulation generally comprising a base vehicle 104, such as safflower oil, and an anti-dysallostatic composition 102. An anti-dysallostatic composition 102 is envisioned herein to counteract the persistent dysallostatic adaptations that a person experiences both physiologically and psychologically to continued use of cannabinoid compounds (continued use, i.e., consumption of cannabinoid compounds by the person, is considered to be at least two uses, but generally more than 10 uses wherein each use is at a frequency that provides a sustained therapeutic effect for the person consuming the cannabinoid compounds). The present embodiment illustratively shows the formulation comprising the base vehicle 104 being approximately 85% and the anti-dysallostatic composition 102 making up the remainder (approximately 15% of the formulation). Other embodiments envision the base vehicle in a range between 50% and 95% of the formulation with the remainder being an anti-dysallostatic composition. As pointed out previously, certain embodiments contemplate the base vehicle being a food-grade oil, such as canola oil, olive oil, seed oil (grape seed, sunflower seed, sesame seed, etc.), peanut oil, safflower oil, coconut oil, palm kernel oil, medium chain triglyceride (MCT) oils, vegetable glycerin, propylene glycol, cocoa butter, etc., or some combination thereof.

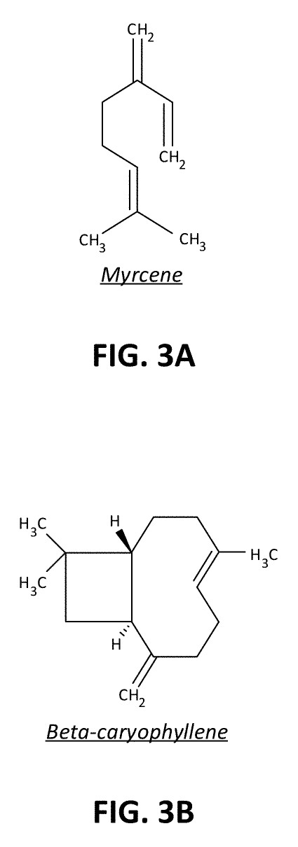

With reference to the anti-dysallostatic composition embodiment 102, the composition 102 can comprise a first part phytoterpenoid and phytocannabinoid 204 and a second part independently derived terpenoid 202, which in one embodiment is a phyterpenoid (plant derived) and in another embodiment is a synthetic terpenoid (synthesize terpenes can be produced with a chemical reaction in a beaker or by genetically engineered bacteria or yeast to produce the terpene, just to name several examples). As shown in FIG. 2, the anti-dysallostatic composition 102 includes a first part 204 comprising phytoterpenoid and phytocannabinoid between 70% and 95% and the second part 202 being an independently derived terpenoid (or terpene) or phytoterpene making up the balance. The balance as used herein is an additional part/portion of a composition or formulation making the total come out to 100%. Hence, if a first part of a composition is 70% then the balance is 30%. Likewise, if a first part of a composition is 85% then the balance is 15%. The independently derived terpenoid 202 unnaturally adds to the amount/quantity of phytoterpenoid found in a cannabis plant. The independently derived terpenoid 202 contributes to the anti-dysallostatic effect. A terpene is a naturally occurring hydrocarbon based on combinations of the isopropane unit and terpenoids are compounds related to the terpenes, which may include some additional functional groups (oxygen functionality or some rearrangement that make up chemicals unique like alcohols, esters, carboxylic acid, etc.), however the two terms can be used interchangeably here. Terpenes are a large and diverse class of organic compounds produced by the cannabis plant/s. Terpenes as used in the anti-dysallostatic composition 102 provides, inter alia, fragrances that associate with the cannabinoids.

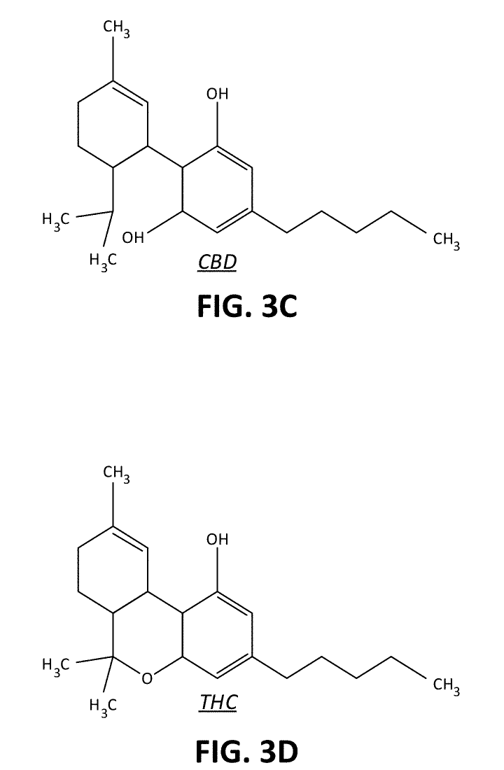

A person suffering from dysallostasis due to extensive use of cannabinoids will once again experience the therapeutic effects of the cannabinoids before dysallostasis with the independently derived added terpenes 202 due to the added effect pharmacological or aromatic scent processed by olfactory systems or taste receptors in the mouth or gut. Generally speaking, terpenes 202 in the present invention can comprise beta-caryophyllene and myrcene. Some embodiments envision the composition of the independently derived terpenoid 202 comprised of between 40% and 60% beta-caryophyllene while the rest is myrcene, yet other embodiments envision the independently derived terpenoid 202 being comprised of approximately 50% beta-caryophyllene and 50% myrcene. The structure of myrcene is shown in FIG. 3A and beta-caryophyllene is shown in FIG. 3B. Certain embodiments envision the first part 204 comprising between 90% and 95% phytocannabinoid and the balance being a phytoterpenoid. More commonly, the largest proportion of phytoterpenoid found in a cannabis plant is approximately 8% with the balance (92%) comprising phytocannabinoids. Hence, one embodiment accepts “natural phytocannabinoid/phytoterpenoid concentration” defining the first part 204 as having a concentration of 92% phytocannabinoid and 8% phytoterpenoids.

Certain embodiments contemplate the independently derived terpenoid 202 being in a range between 5% and 20% of the anti-dysallostatic composition 102, while other embodiments envision the independently derived terpenoid 202 being in a range between 5% and 20% of the anti-dysallostatic composition 102. Certain commercial embodiments envision producing a base vehicle 104 that is at least 90% of the formulation while the rest is the anti-dysallostatic 102. Phytocannabinoids in the present embodiment can comprise approximately 20% CBD (Cannabidiol) and 80% THC (Tetrahydrocannabinol) as illustratively shown in FIGS. 3C and 3D, respectively. Other concentrations of CBD and THC are contemplated along with a variety of other cannabinoids that may be present in the mixture of phytocannabinoids. THC is one of at least 113 cannabinoids identified in cannabis. THC is the principal psychoactive constituent of cannabis. With chemical name (−)-trans-Δ⁹-tetrahydrocannabinol, the term THC also refers to cannabinoid isomers. CBD is widely recognized not to be psychoactive, generally free of terpenes and can account up to 40% of cannabis extract.

Certain embodiments of the present invention envision deployment of the formulation to an adult human subject in measured doses to manage dysallostasis. Formulation delivery can include a number of deployment options such as a sublingual spray or eyedropper, oral or suppository capsule, nasal spray, vape system, etc. Different dosage amounts of the formulation can be dependent on the delivery method. For example, for sublingual delivery a single dose of the anti-dysallostatic composition 102 is envisioned in one embodiment to comprise approximately 5 mg of THC, approximately 1.25 mg of CBD and between 0.3-0.6 of the phytoterpenoid (e.g., the “natural phytocannabinoid/phytoterpenoid concentration”) with the with the remainder comprising the independently derived terpenoid 202 having a quantifiable amount of 0.3-2.0 mg. Certain other embodiments contemplate that as little as 0.1 mg/kg of body weight can halt the effects of dysallostasis. As one skilled in the art will recognize a single dose of the anti-dysallostatic composition 102 may contain a significant portion being the base vehicle 104. The base vehicle in certain delivery scenarios is envisioned to have minimal effects on dosage absorption into the human body.

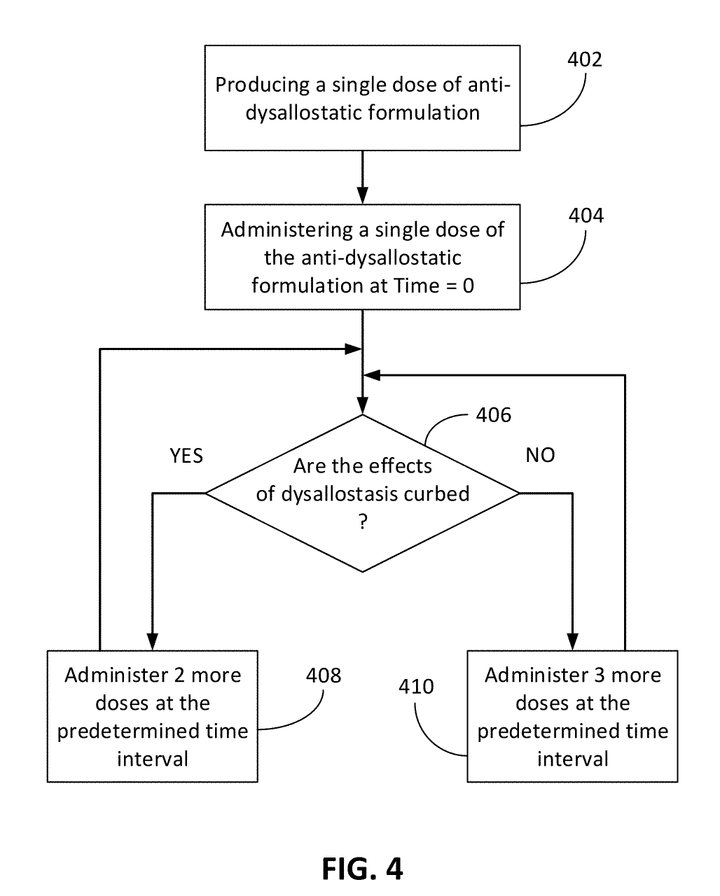

FIG. 4 shows a block diagram of a method to use the formulation 100 to address the effects of dysallostasis in a person/subject due to repeated cannabinoid administration consistent with embodiments of the present invention. As shown in step 402, a single dose of anti-dysallostatic composition 102 (which can be established within the scope of the embodiments described above) is produced by a delivery system, such as a spray device, an eyedropper, a pill, etc. Initially administer a single dose of the anti-dysallostatic composition 102 to the subject while they are in a state of dysallostasis from repeated administration of cannabinoids, step 404. Administering the single dose can be accomplished by way of a number of pathways including, but not limited to, sublingual introduction. As shown in decisions step 406, determine if the subject is no longer in a state of dysallostasis after a predetermined amount of time following the initial dose of step 404. Determining if a subject is no longer in a state of dysallostasis can be either objectively evaluated (through analytical devices, for example) or subjectively evaluated by asking the person/subject if they are experiencing positive effects of the dose or doses. Certain embodiments envision the predetermined amount of time being between 4 hours and 10 hours, while other embodiments envision between 6 hours and 10 hours. If the subject is no longer in a state of dysallostasis after the predetermined amount of time following the initial dose, administer two more doses to the subject, preferably in the administration technique initially used, step 408. If the subject is still in a state of dysallostasis after the predetermined amount of time following the initial dose, administer three more doses to the subject, preferably in the administration technique initially used, step 410. With continued reference to step 408, after administering the two doses of the anti-dysallostatic composition 102, return to step 406 to reevaluate and repeat. Reevaluation can be shortly following administration of the two or more doses (1 minute to 15 minutes for example), or can be at the predetermined amount of time defined by the time interval, or optionally somewhere in between. With continued reference to step 408, after administering the three doses of the anti-dysallostatic composition 102, return to step 406 to reevaluate and repeat. Certain embodiments envision increasing to 4 doses and beyond if the effects of dysallostasis do not subside after the administration of three more doses in step 410.

One method of producing the anti-dysallostatic composition 102 can include obtaining a first cultivated cannabis strain possessing a greater than 15% THC with only trace amounts of other cannabinoids, such as under 0.5%. Obtaining a second cultivated cannabis strain possessing over 5% CBD with only trace amounts of other cannabinoids under 0.5%. The THC and the CBD can then be extracted by way of using carbon dioxide as a solvent. The THC and the CBD extracts can then be winterized using ethanol at approximately 50° F. for 24 hours to separate fats and lipids from the cannabinoids. Beta-caryophyllene and myrcene are added to the THC and CBD carrier liquid to the appropriate concentrations.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms used herein. For example, though embodiments of the present invention describe beta-caryophyllene and myrcene as isolated combinations of terpenes, other terpenes may be beneficial and can be used with or in substitution of without departing from the scope and spirit of the present invention. The specification and drawings are to be regarded as illustrative and exemplary rather than restrictive. For example, the word “preferably,” and the phrase “preferably but not necessarily,” are used synonymously herein to consistently include the meaning of “not necessarily” or optionally. “Comprising,” “including,” and “having,” are intended to be open-ended terms.

It will be clear that the claimed invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the claimed invention disclosed and as defined in the appended claims. Accordingly, it is to be understood that even though numerous characteristics and advantages of various aspects have been set forth in the foregoing description, together with details of the structure and function, this disclosure is illustrative only, and changes may be made in detail, especially in matters of structure and arrangement to the full extent indicated by the broad general meaning of the terms in which the appended claims are expressed.

What is claimed is:

1. A formulation to prevent dysallostasis effects of physiological responses to cannabinoids from repeated administration, the formulation comprising:
a base vehicle and an anti-dysallostatic composition, the anti-dysallostatic composition comprising a first part phytoterpenoid and phytocannabinoid and a second part independently derived phytoterpenoid, the first part is between 70% and 95%.

2. The formulation of claim 1 wherein the independently derived phytoterpenoid is in a range between 5% and 20% of the anti-dysallostatic composition.

3. The formulation of claim 1 wherein the independently derived phytoterpenoid is in a range between 5% and 10% of the anti-dysallostatic composition.

4. The formulation of claim 1 wherein the independently derived phytoterpenoid is comprised of approximately 50% beta-caryophyllene and 50% myrcene.

5. The formulation of claim 1 wherein the independently derived phytoterpenoid is comprised of between 40% and 60% beta-caryophyllene and the rest is myrcene.

6. The formulation of claim 1 wherein the base vehicle is at least 90% and the anti-dysallostatic composition comprises the rest of the formulation.

7. The formulation of claim 1 wherein the phytocannabinoid comprises approximately 20% CBD and 80% THC.

8. The formulation of claim 1 wherein a single dose of the phytocannabinoid comprises approximately 5 mg of THC and approximately 1 mg of CBD.

9. The formulation of claim 1 wherein the phytoterpenoid and phytocannabinoid is essentially composed of approximately 92% phytocannabinoid and 8% phytoterpenoid.

10. The formulation of claim 1 wherein the base vehicle is a plant derived oil.

11. The formulation of claim 1 wherein the base vehicle is between 50% and 94% of the formulation.

12. A method for managing dysallostasis effects to cannabinoids from repeated administration, the method comprising:
providing an anti-dysallostatic composition essentially comprised of a combination of phytoterpenoid and phytocannabinoid plus an independently derived phytoterpenoid, the combination of phytoterpenoid and phytocannabinoid is between 70% and 95%, the phytocannabinoid includes THC and CBD;
introducing a dose of the composition sublingually to a recipient in a state of dysallostasis from prior consumption of the THC, the dose is approximately 5 mg of the THC, 1 mg of the CBD and 0.3-0.6 mg of the phytoterpenoid and between approximately 0.3-2.0 mg of the independently derived phytoterpenoid; and
if there is a change from the state of dysallostasis after the introducing step, increasing administration of the composition sublingually to two of the doses at a predetermined time interval, if there is not a change from the state of dysallostasis after the introducing step, increasing administration of the composition sublingually to 3 of the doses at the predetermined interval.

13. The method of claim 12 wherein the predetermined time interval is between 6 and 10 hours.

14. The method of claim 12 wherein the introducing step is accomplished sublingually.

15. The method of claim 12 wherein the anti-dysallostatic composition is combined with a base vehicle.

16. The method of claim 15 wherein the base vehicle is a plant derived oil that is between 50% and 94% of the formulation.

17. The method of claim 12 wherein the independently derived phytoterpenoid is comprised of approximately 50% beta-caryophyllene and 50% myrcene.

18. A formulation to prevent dysallostasis effects of physiological responses to cannabinoids from repeated administration, the formulation comprising:
a dose of an anti-dysallostatic composition adapted to be administered to a recipient sublingually while the recipient is in a state of dysallostasis from prior consumption of THC, the dose is approximately 5 mg of the THC, 1 mg of the CBD, 0.3-0.6 mg of the phytoterpenoid and between approximately 0.3-2.0 mg of the independently derived terpenoid, the phytoterpenoid is essentially composed of beta-caryophyllene and myrcene; and
a plant derived oil adapted to be a base vehicle for the dose.

19. The formulation of claim 18 wherein at least one of the doses is adapted to be administered to the recipient at regular time intervals.

20. The formulation of claim 18 wherein the terpenoid is and independently derived phytoterpenoid.
